SEQUENCE LISTING (9 sequences)

SEQ ID NO: 1 - protein, 366 aa, Artificial Sequence
  Full-length CA-TCF1
MYKETVYSAFNLLMHYPPPSGAGQHPQPQPPLHKANQPPHGVPQLSLYEHFNSPHPTPAPADISQKQVHRPLQTPDLSGFYSLTSGSMGQLPHTVSWFTHPSLMLGSGVPGHPAAIPHPAIVPPSGKQELQPFDRNLKTQAESKAEKEAKKPTIKKPLNAFMLYMKEMRAKVIAECTLKESAAINQILGRRWHALSREEQAKYYELARKERQLHMQLYPGWSARDNYGKKKRRSREKHQESTTETNWPRELKDGNGQESLSMSSSSSPAGGGGSGGGGSDLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 2 - protein, 269 aa, Artificial Sequence
  TCF7 domain (isoform 4S)
MYKETVYSAFNLLMHYPPPSGAGQHPQPQPPLHKANQPPHGVPQLSLYEHFNSPHPTPAPADISQKQVHRPLQTPDLSGFYSLTSGSMGQLPHTVSWFTHPSLMLGSGVPGHPAAIPHPAIVPPSGKQELQPFDRNLKTQAESKAEKEAKKPTIKKPLNAFMLYMKEMRAKVIAECTLKESAAINQILGRRWHALSREEQAKYYELARKERQLHMQLYPGWSARDNYGKKKRRSREKHQESTTETNWPRELKDGNGQESLSMSSSSSPA

SEQ ID NO: 3 - DNA, 10 bp, Artificial Sequence
  Glycine serine linker
ggggsggggs

SEQ ID NO: 4 - protein, 87 aa, Artificial Sequence
  beta-catenin trans-activation domain (amino acids 688-774)
DLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 5 - DNA, 1098 bp, Artificial Sequence
  Full-length CA-TCF1
atgtacaaagagaccgtttattccgcgtttaacctcttgatgcattaccctccaccctctggtgccggacaacacccacaaccgcaacctcccttgcacaaagctaatcaaccgccgcatggcgtcccgcaactctctctctatgaacacttcaactctccacacccaacccccgcacctgcggacatttctcaaaaacaggttcatcgacctttgcaaacacctgacctgtcaggcttttactcactgactagcggttccatgggtcaattgccacacacagtaagctggttcacgcatccctcactcatgcttgggtccggcgtcccaggacaccccgcggccattccccatcctgccatcgttccgccatccggtaaacaggaactgcagccatttgaccggaatctcaagacgcaagcagagtccaaggctgagaaggaggctaagaagcccacgataaaaaaaccgttgaatgcgtttatgttgtacatgaaagagatgagggctaaggtaatagcggaatgcacattgaaggagtccgctgccatcaaccagatactgggtagacgatggcatgcgttgtcacgcgaggagcaagcgaaatattatgagctggctagaaaggagaggcagctccacatgcaattgtatcccggttggagcgcgagggacaactacggcaagaaaaagagacggtctagggaaaaacaccaagaaagtactacggagacgaattggcccagggaacttaaggacggcaatgggcaagaatctcttagtatgtcatctagcagctctccggccggtggcggaggttccggcggaggcgggtctgatctgggccttgacataggagcccaaggggaaccgcttgggtatcgacaagatgatccttcataccggagtttccattctggaggttatgggcaagacgcccttggtatggaccctatgatggagcacgagatgggcggccatcaccctggagctgattacccagtcgatggcttgcctgacctgggccatgcccaggaccttatggatggcctcccacccggggattccaaccagctggcgtggttcgacactgacctt

SEQ ID NO: 6 - DNA, 807 bp, Artificial Sequence
  TCF7 domain (Isoform 4S)
atgtacaaagagaccgtttattccgcgtttaacctcttgatgcattaccctccaccctctggtgccggacaacacccacaaccgcaacctcccttgcacaaagctaatcaaccgccgcatggcgtcccgcaactctctctctatgaacacttcaactctccacacccaacccccgcacctgcggacatttctcaaaaacaggttcatcgacctttgcaaacacctgacctgtcaggcttttactcactgactagcggttccatgggtcaattgccacacacagtaagctggttcacgcatccctcactcatgcttgggtccggcgtcccaggacaccccgcggccattccccatcctgccatcgttccgccatccggtaaacaggaactgcagccatttgaccggaatctcaagacgcaagcagagtccaaggctgagaaggaggctaagaagcccacgataaaaaaaccgttgaatgcgtttatgttgtacatgaaagagatgagggctaaggtaatagcggaatgcacattgaaggagtccgctgccatcaaccagatactgggtagacgatggcatgcgttgtcacgcgaggagcaagcgaaatattatgagctggctagaaaggagaggcagctccacatgcaattgtatcccggttggagcgcgagggacaactacggcaagaaaaagagacggtctagggaaaaacaccaagaaagtactacggagacgaattggcccagggaacttaaggacggcaatgggcaagaatctcttagtatgtcatctagcagctctccggcc

SEQ ID NO: 7 - DNA, 30 bp, Artificial Sequence
  Glycine serine linker
ggtggcggaggttccggcggaggcgggtct

SEQ ID NO: 8 - DNA, 261 bp, Artificial Sequence
  beta-Catenin trans-activation domain
gatctgggccttgacataggagcccaaggggaaccgcttgggtatcgacaagatgatccttcataccggagtttccattctggaggttatgggcaagacgcccttggtatggaccctatgatggagcacgagatgggcggccatcaccctggagctgattacccagtcgatggcttgcctgacctgggccatgcccaggaccttatggatggcctcccacccggggattccaaccagctggcgtggttcgacactgacctt

SEQ ID NO: 9 - DNA, 63 bp, Artificial Sequence
  T2A
ggcggcggagagggcagaggaagtcttctaacatgcggtgacgtggaggagaatcccggccct

(19) United States
(12) Patent Application Publication
(10) Pub. No.: US 2023/0303643 A1
(43) Pub. Date: Sep. 28, 2023

(54) CONSTITUTIVELY ACTIVE TCF1 TO PROMOTE MEMORY-ASSOCIATED TRAITS IN CAR T CELLS

(71) Applicant: Seattle Children's Hospital (dba Seattle Children's Research Institute), Seattle, WA (US)

(72) Inventors: Christopher P. Saxby, Seattle, WA (US); Michael C. Jensen, Seattle, WA (US)

(21) Appl. No.: 18/021,450

(22) PCT Filed: Aug. 18, 2021
(86) PCT No.: PCT/US2021/046449

§ 371 Date: Feb. 15, 2023

Related U.S. Application Data
(60) Provisional application No. 63/068,566, filed on Aug. 21, 2020.

(51) Int. Cl. C07K 14/47

(52) U.S. Cl. CPC C07K 14/4702; C07K 2319/00

(57) ABSTRACT

Some embodiments of the methods and compositions provided herein relate to chimeric proteins comprising a T cell factor 1 (TCF1) domain and a β-catenin transactivation domain. In some embodiments, populations of cells containing such chimeric proteins have an increased level of memory T cell associated markers and activities compared to populations lacking the chimeric proteins. Some embodiments include populations of cells comprising the chimeric proteins and chimeric antigen receptors and uses thereof.

### CROSS-REFERENCE TO RELATED APPLICATIONS

This application claims priority to U.S. Prov. App. No. 63/068,566 filed Aug. 21, 2020 entitled “CONSTITUTIVELY ACTIVE TCF1 TO PROMOTE MEMORY-ASSOCIATED TRAITS IN CAR T CELLS” which is hereby expressly incorporated by reference herein in its entirety.

### REFERENCE TO SEQUENCE LISTING

The present application is being filed along with a Sequence Listing in electronic format. The Sequence Listing is provided as a file entitled SCRI338WOSEQLIST.TXT, created Jul. 20, 2021, which is approximately 10 Kb in size. The information in the electronic format of the Sequence Listing is incorporated herein by reference in its entirety.

### FIELD OF THE INVENTION

Some embodiments of the methods and compositions provided herein relate to chimeric proteins comprising a T cell factor 1 (TCF1) domain and a β-catenin transactivation domain. In some embodiments, populations of cells containing these chimeric proteins, such as cells having chimeric antigen receptors (CARs) comprising these proteins, exhibit an increased level of memory T cell associated markers and related activities, as compared to cell populations lacking these chimeric proteins or CARs. Accordingly, embodiments described herein include populations of cells comprising these chimeric proteins and chimeric antigen receptors and uses of these populations of cells to treat or inhibit diseases such as a cancer including but not limited to colon, lung, liver, breast, renal, prostate, ovarian, skin bone, leukemia, multiple myeloma, or brain cancer.

### BACKGROUND OF THE INVENTION

In cell-based adoptive immunotherapy, T cells isolated from a patient can be modified to express synthetic proteins that enable the cells to perform new therapeutic functions after they are subsequently transferred back into the patient. Examples of such synthetic proteins are chimeric antigen receptors (CARs) and engineered T cell Receptors (TCR). An example of a currently used CAR is a fusion of an extracellular recognition domain (e.g., an antigen-binding domain), a transmembrane domain, and one or more intracellular signaling domains. Upon antigen engagement, the intracellular signaling portion of the CAR can initiate an activation-related response in an immune cell, such as release of cytolytic molecules to induce tumor cell death. However, there is a continued need for improved cell-based adoptive immunotherapies SUMMARY OF THE INVENTION

Some embodiments of the methods and compositions provided herein include a polynucleotide encoding a chimeric polypeptide comprising a first nucleic acid encoding a T cell factor 1 (TCF1) domain and a second nucleic acid encoding a β-catenin transactivation domain.

In some embodiments, the TCF1 domain comprises a TCF1 isoform 4S.

In some embodiments, the TCF1 domain comprises or consists of an amino acid sequence having at least 90%, 91%, 92%, 93%, 94%, 95%, 96%, 97%, 98%, 99%, or 100% sequence identity to SEQ ID NO:02. In some embodiments, the TCF1 domain comprises or consists of the amino acid sequence of SEQ ID NO:02.

In some embodiments, the β-catenin transactivation domain comprises or consists of an amino acid sequence having at least 90%, 91%, 92%, 93%, 94%, 95%, 96%, 97%, 98%, 99%, or 100% sequence identity to SEQ ID NO:04. In some embodiments, the p-catenin transactivation domain comprises or consists of the amino acid sequence of SEQ ID NO:04.

In some embodiments, the TCF1 domain is linked to the β-catenin transactivation domain via a linker. In some embodiments, the linker has a length in a range from 2 residues to 20 residues, such as 2, 3, 4, 5, 6, 7, 8, 9, 10, 11, 12, 13, 14, 15, 16, 17, 18, 19, or 20 residues or a length that is within a range defined by any two of the aforementioned lengths. In some embodiments, the linker has a length of 10 residues. In some embodiments, the linker comprises or consists of the amino acid sequence of SEQ ID NO:03.

In some embodiments, the chimeric polypeptide comprises or consists of an amino acid sequence having at least 90%, 91%, 92%, 93%, 94%, 95%, 96%, 97%, 98%, 99%, or 100/6 sequence identity to SEQ ID NO:01. In some embodiments, the chimeric polypeptide comprises or consists of the amino acid sequence of SEQ ID NO:01.

Some embodiments also include a third nucleic acid encoding a cell surface selectable marker. In some embodiments, the cell surface selectable marker is selected from a truncated HER2 (Her2tG) polypeptide, a truncated EGFR (EGFRt) polypeptide, or a truncated CD19 (CD19t).

In some embodiments, the second nucleic acid and the third nucleic acid are linked via a ribosomal skip sequence. In some embodiments, the ribosomal skip sequence is selected from P2A, T2A, E2A or F2A.

Some embodiments also include an inducible promoter operably linked to the first nucleic acid.

Some embodiments also include an inducible cytotoxic gene. In some embodiments, the cytotoxic gene encodes a protein selected from a thymidine kinase, thymidine kinase fused to thymidylate kinase, oxidoreductase, deoxycytidine kinase, uracil phosphoribosyltransferase, cytosine deaminase, or cytosine deaminase fused to uracil phosphonbosyltransferase. In some embodiments, the cytotoxic gene encodes a thymidine kinase.

Some embodiments of the methods and compositions provided herein include a vector comprising any one of the polynucleotides provided herein. In some embodiments, the vector comprises a viral vector. In some embodiments, the vector is selected from a lentiviral vector, an adeno-associated viral vector, or an adenoviral vector. In some embodiments, the vector comprises a lentiviral vector.

Some embodiments of the methods and compositions provided herein include a polypeptide encoded by any one of the polynucleotides provided herein.

Some embodiments of the methods and compositions provided herein include a cell comprising any one of the polynucleotides provided herein.

Some embodiments also include a fourth nucleic acid encoding a chimeric antigen receptor (CAR), or a CAR protein.

In some embodiments, the CAR is capable of binding or configured to bind specifically to a target antigen expressed by a cancer cell. In some embodiments, the cell is a T cell. In some embodiments, the cell is derived from a CD4+ T cell, a CD8+ T cell, a precursor T cell, or a hematopoietic stem cell. In some embodiments, the CD8+ T cell is a CD8+ cytotoxic T lymphocyte cell selected from the group consisting of a naïve CD8+ T cell, a central memory CD8+ T cell, an effector memory CD8+ T cell, and a bulk CD8+ T cell. In some embodiments, the CD4+ cell is a CD4+ helper T lymphocyte cell selected from the group consisting of a naïve CD4+ T cell, a central memory CD4+ T cell, an effector memory CD4+ T cell, and a bulk CD4+ T cell. In some embodiments, the cell is a primary cell. In some embodiments, the cell is mammalian. In some embodiments, the cell is human. In some embodiments, the cell is ex vivo.

Some embodiments of the methods and compositions provided herein include a pharmaceutical composition comprising any one of the cells provided herein and a pharmaceutically acceptable excipient.

Some embodiments of the methods and compositions provided herein include a method of treating, ameliorating or inhibiting a disorder in a subject, comprising: administering any one of the cells provided herein to the subject in need thereof, wherein the cell comprises a chimeric antigen receptor (CAR); optionally wherein said subject is selected or identified to receive a medicament for said disorder, such as by clinical or diagnostic evaluation for the presence of said disorder. In some embodiments, the disorder comprises a cancer comprising a target antigen, wherein the CAR is capable of binding or is configured to bind specifically to the target antigen. In some embodiments, the cancer is selected from a solid tumor such as a colon cancer, breast cancer, ovarian cancer, lung cancer, pancreatic cancer, prostate cancer, melanoma, renal cancer, pancreatic cancer, brain cancer, glioblastoma, neuroblastoma, medulloblastoma, sarcoma, bone cancer, or liver cancer, or a non-solid tumor, such as a leukemia, or a multiple myeloma. In some embodiments, the cell is autologous to the subject. In some embodiments, the cell is not autologous to the subject. In some embodiments, the subject is mammalian. In some embodiments, the subject is human.

Some embodiments of the methods and compositions provided herein include any one of the cells provided herein for use in treating, ameliorating or inhibiting a disease in a subject, such as a cancer, or a disorder stemming from said disease.

Some embodiments of the methods and compositions provided herein include the use of any one of the cells provided herein as a medicament, such as for use in treating, ameliorating or inhibiting a disease, such as a cancer or a disorder stemming from said disease in a subject.

Some embodiments of the methods and compositions provided herein include a method of preparing a population of T cells comprising introducing any one of the polynucleotides provided herein into a T cell to obtain a transduced T cell and culturing the transduced T cell to obtain a population of T cells, wherein the population of T cells has an increased level of a memory T cell marker compared to a population of T cells lacking the polynucleotide. In some embodiments, the memory T cell marker is selected from CCR7, CD62L, CD127, CD45RO, CD44, CD27, CD28, CD95, CXCR3, or LFA-1.

In some embodiments, the T cell is derived from a CD4+ T cell, a CD8+ T cell, a precursor T cell, or a hematopoietic stem cell. In some embodiments, the CD8+ T cell is a CD8+ cytotoxic T lymphocyte cell selected from the group consisting of a naïve CD8+ T cell, a central memory CD8+ T cell, an effector memory CD8+ T cell, and a bulk CD8+ T cell. In some embodiments, the CD4+ cell is a CD4+ helper T lymphocyte cell selected from the group consisting of a naïve CD4+ T cell, a central memory CD4+ T cell, an effector memory CD4+ T cell, and a bulk CD4+ T cell. In some embodiments, the cell is a primary cell. In some embodiments, the cell is mammalian. In some embodiments, the cell is human. In some embodiments, the cell is ex vivo.

### BRIEF DESCRIPTION OF THE DRAWINGS

FIG. 1 depicts a schematic view of a transgene encoding a TCF1 domain, a glycine-serine linker (G4S)2, a β catenin transactivator domain (BCatTA), a T2A ribosome skip sequence, and truncated HER2 polypeptide (Her2G).

FIG. 2 depicts a graph of relative luminescence for cells transfected with expression vectors encoding either: a cell surface marker (Her2tG), a TCF1 domain linked to Her2tG (TCF1-Her2tG), or CA-TCF1 linked to Her2tG. CA-TCF1 includes a TCF1 domain, linked to p catenin transactivation domain.

FIG. 3A depicts a FACS analysis of CD8+ T cells expressing CA-TCF1 and stained for memory-associated surface markers CCR7 and CD62L.

FIG. 3B depicts a graph relative levels of memory cell-associated surface markers CCR7 and CD62L in CD8+ T cells expressing CA-TCF1 and stained for CCR7 and CD62L.

### DETAILED DESCRIPTION

T cell factor 1 (TCF1) is responsible for preserving the CD8+ T cell response to chronic viral infections (see Utzschneider, D. T. et al. (2016) Immunity 45, 415-427; and Wang, Y. et al. (2019) Front. Immunol. 10, 1-14). Furthermore, TCF1 is elevated in CAR T cell groups responsible for better outcomes in patients with chronic lymphocytic leukemia (see Fraietta, J. A. et al. (2018) Nat. Med. doi:10.1038/s41591-018-0010-1). TCF1 promotes a memory T cell-associated phenotype, and CAR T cells generated from memory T cells provide superior anti-tumor activity as compared to other T cell subsets (see Muralidharan, S. et al. (2011) J. Immunol. 187, 5221-32; and Sommermeyer, D. et al. (2016) Leukemia 30, 492-500).

Expression of wild-type TCF1 alone may not be sufficient to confer the benefits of memory T cell-associated traits to CAR T cell therapy. In the canonical Wnt signaling pathway, TCF1 becomes activated upon association with β-catenin, which then recruits transcriptional coregulator proteins to initiate transcription (see Eisenmann, D. M. Wnt signaling. WormBook 1-17 (2005). doi:10.1895/wormbook.1.7.1; and Vleminckx, K., et al. (1999) Mech. Dev. 81, 65-74). Thus, in order for TCF1-dependent transcription to occur, cells must contain sufficient levels of TCF1 and β-catenin. Indeed, overexpression of both TCF1 and β-catenin, but neither alone, in mouse T cells resulted in increased memory T cell differentiation in response to bacterial infection (see Zhao, D.-M. et al. (2011) J. Immunol. 184, 1191-1199).

The difficulty of introducing transgenes into T cells increases with the size and number of the genes introduced. Specifically, the efficiency of transgene introduction decreases, leading to higher costs and potentially poorer T cell products as the purification of modified populations becomes more cumbersome. Introducing both TCF1 and β-catenin into a cell encoded as separate polypeptides requires a DNA footprint of 3495 base pairs. In contrast, a transgene that includes a TCF1 domain linked to a β-catenin transactivation domain (CA-TCF1) has about 1098 bp. Furthermore, simultaneous expression of TCF1 and β-catenin as separate polypeptides encoded in a single polycistronic transcript requires the use of a ribosomal skip sequence or internal entry site, both of which result in decreased expression of transgenes. In some embodiments, a CA-TCF1 protein is a transcriptionally active unit, whereas separate TCF1 and β-catenin proteins require association to become active. Accordingly, the efficiency of transcriptional activation by a CA-TCF1 is likely higher on a per-protein basis, as it does not depend on a preliminary association reaction to occur.

Current approaches to promote memory T cell differentiation include specific T cell culture conditions and the introduction of other transcription factors that divert T cell differentiation away from exhausted T cell states. Unlike any other technologies, certain embodiments provided herein include constitutively active versions of a transcription factor so as to specifically promote memory T cell differentiation.

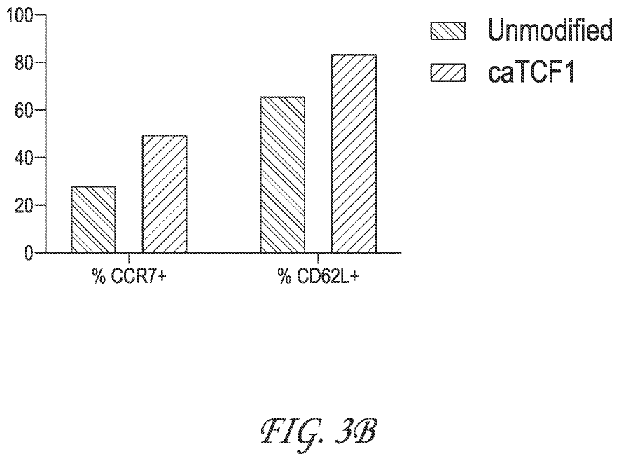

TCF1 promotes the acquisition of memory T cell-associated characteristics and is responsible for sustaining T cell activity under extended periods of antigen exposure. In some embodiments, CA-TCF1 is introduced into CAR T cells to promote these characteristics and subsequently increase the efficacy of the therapy. As described herein, results indicated that T cells expressing CA-TCF1 maintain higher levels of expression of memory-associated surface markers CD62L and CCR7 (see FIG. 3A, FIG. 3B). These findings provide evidence that CA-TCF1 was able to generate transcriptional changes in primary T cells consistent with the established role of TCF1. In some embodiments, TCF1 is applied to a T cell immunotherapy system in which memory T cell characteristics lead to superior or improved outcomes.

Some embodiments provided herein include chimeric proteins and use of these chimeric proteins to supplement chimeric antigen receptor (CAR) therapy. Some embodiments include a constitutively active of TCF1. Some embodiments include a population of CAR T cells having an increased memory T cell-associated phenotype, as compared to a population of CAR T cells lacking a constitutively active of TCF1.

### Definitions

Unless defined otherwise, all technical and scientific terms used herein have the same meaning as commonly understood by one of ordinary skill in the art to which the invention pertains.

As used herein, “a” or “an” may mean one or more than one.

“About” as used herein when referring to a measurable value is meant to encompass variations of ±20% or ±10%, more preferably ±5%, even more preferably ±1%, and still more preferably ±0.1% from the specified value.

As used herein, “nucleic acid” or “nucleic acid molecule” have their plain and ordinary meaning in view of the whole specification and may to refer to, for example, polynucleotides, such as deoxyribonucleic acid (DNA) or ribonucleic acid (RNA), oligonucleotides, fragments generated by the polymerase chain reaction (PCR), or fragments generated by any of ligation, scission, endonuclease action, or exonuclease action. Nucleic acid molecules can be composed of monomers that are naturally occurring nucleotides (such as DNA or RNA), or analogs of naturally occurring nucleotides (e.g., enantiomeric forms of naturally occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, or azido groups, or sugars can be functionalized as ethers or esters. Moreover, the entire sugar moiety can be replaced with sterically and electronically similar structures, such as aza-sugars or carbocyclic sugar analogs. Examples of modifications in a base moiety include alkylated purines or pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate, and the like. The term “nucleic acid molecule” also includes so-called “peptide nucleic acids,” which comprise naturally occurring or modified nucleic acid bases attached to a polyamide backbone. Nucleic acids can be either single stranded or double stranded. In some embodiments, a nucleic acid sequence encoding a fusion protein is provided. In some embodiments, the nucleic acid encoding the CAR specific for CD171 is RNA or DNA.

As used herein, “coding for” or “encoding” has its plain and ordinary meaning when read in light of the specification, and includes, for example, the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system.

As used herein, “chimeric antigen receptor” (CAR) has its plain and ordinary meaning when read in light of the specification, and may include but is not limited to, for example, a synthetically designed receptor comprising a ligand binding domain of an antibody or other protein sequence that binds to a molecule associated with a disease or disorder and is, preferably, linked via a spacer domain to one or more intracellular signaling domains of a cell, such as a T cell, or other receptors, such as one or more costimulatory domains. Chimeric receptor can also be referred to as artificial cell receptors or T cell receptors, chimeric cell receptors or T cell receptors, chimeric immunoreceptors, or CARs. These receptors can be used to graft the specificity of a monoclonal antibody or binding fragment thereof onto a cell, preferably a T-cell, with transfer of their coding sequence facilitated by viral vectors, such as a retroviral vector or a lentiviral vector. CARs can be, in some instances, genetically engineered T cell receptors designed to redirect T cells to target cells that express specific cell-surface antigens. T cells can be removed from a subject and modified so that they can express receptors that can be specific for an antigen by a process called adoptive cell transfer. The T cells are reintroduced into the patient where they can then recognize and target an antigen. CARs are also engineered receptors that can graft an arbitrary specificity onto an immune receptor cell. CARs are considered by some investigators to include the antibody or antibody fragment, preferably an antigen binding fragment of an antibody, the spacer, signaling domain, and transmembrane region. Due to the surprising effects of modifying the different components or domains of the CAR described herein, such as the epitope binding region (for example, antibody fragment, scFv, or portion thereof), spacer, transmembrane domain, and/or signaling domain), the components of the CAR are frequently distinguished throughout this disclosure in terms of independent elements. The variation of the different elements of the CAR can, for example, lead to a desired binding affinity, such as a stronger binding affinity for a specific epitope or antigen.

The CARs graft the specificity of a monoclonal antibody or binding fragment thereof or scFv onto a T cell, with the transfer of their coding sequence facilitated by vectors. In order to use CARs as a therapy for a subject in need, a technique called adoptive cell transfer is used in which T cells are removed from a subject and modified so that they can express the CARs that are specific for an antigen. The T cells, which can then recognize and target an antigen, are reintroduced into the patient.

As used herein, a “ribosome skip sequence” has its plain and ordinary meaning when read in light of the specification, and includes, for example, a sequence that during translation, forces the ribosome to “skip” the ribosome skip sequence and translate the region after the ribosome skip sequence without formation of a peptide bond. Several viruses, for example, have ribosome skip sequences that allow sequential translation of several proteins on a single nucleic acid without having the proteins linked via a peptide bond. As described herein, this is the “linker” sequence. In some alternatives of the nucleic acids provided herein, the nucleic acids comprise a ribosome skip sequence between the sequence for the chimeric antigen receptor and the sequence of the marker protein, such that the proteins are co-expressed and not linked by a peptide bond. In some embodiments, the ribosome skip sequence is a P2A, T2A, E2A or F2A sequence.

As used herein, a “marker sequence,” has its plain and ordinary meaning when read in light of the specification, and includes, for example, a protein that is used for selecting or tracking a protein or cell that has a protein of interest. In the alternatives described herein, the fusion protein provided can comprise a marker sequence that can be selected in experiments, such as flow cytometry. In some embodiments, the marker comprises a truncated Her2 (Her2t) polypeptide, or a truncated EGFR (EGFRt).

As used herein, “suicide gene therapy,” “suicide genes” and “suicide gene systems” have their plain and ordinary meaning when read in light of the specification, and includes, for example, methods to destroy a cell through apoptosis, which requires a suicide gene that will cause a cell to kill itself by apoptosis. Due to safety concerns for the patients in need of using genetically modified immune cells for treatment or modification of a tumor environment, strategies are being developed in order to prevent or abate adverse events. Adverse effects of incorporation of genetically modified immune cells into a subject for a pretreatment step can include “cytokine storms,” which is a cytokine release syndrome, wherein the infused T-cells release cytokines into the bloodstream, which can lead to dangerously high fevers, as well as a precipitous drop in blood pressure. Control of the system by tamoxifen, as previously described, may also be used when there is indication of a cytokine storm, such as a fever.

As used herein, “vector” or “construct” has its plain and ordinary meaning when read in light of the specification, and includes, for example, a nucleic acid used to introduce heterologous nucleic acids into a cell that has regulatory elements to provide expression of the heterologous nucleic acids in the cell. Vectors include but are not limited to plasmid, minicircles, yeast, viral genomes, lentiviral vector, foamy viral vector, retroviral vector or gammaretroviral vector. The vector may be DNA or RNA, such as mRNA.

As used herein, “T-cells” or “T lymphocytes” can be from any mammal, preferably a primate, including monkeys or humans, a companion animal such as a dog, cat, or horse, or a domestic animal, such as sheep, goats, or cattle. In some alternatives the T-cells are allogeneic (from the same species but different donor) as the recipient subject; in some alternatives the T-cells are autologous (the donor and the recipient are the same): in some alternatives the T-cells are syngeneic (the donor and the recipients are different but are identical twins).

As used herein, “T cell precursors” refers to lymphoid precursor cells that can migrate to the thymus and become T cell precursors, which do not express a T cell receptor. All T cells originate from hematopoietic stem cells in the bone marrow. Hematopoietic progenitors (lymphoid progenitor cells) from hematopoietic stem cells populate the thymus and expand by cell division to generate a large population of immature thymocytes. The earliest thymocytes express neither CD4 nor CD8 and are therefore classed as double-negative (CD4−CD8−) cells. As they progress through their development, they become double-positive thymocytes (CD4+CD8+), and finally mature to single-positive (CD4+CD8− or CD4−CD8+) thymocytes that are then released from the thymus to peripheral tissues.

As used herein, “hematopoietic stem cells” or “HSC” are precursor cells that can give rise to myeloid cells such as, for example, macrophages, monocytes, macrophages, neutrophils, basophils, eosinophils, erythrocytes, megakaryocytes/platelets, dendritic cells and/or lymphoid lineages (such as, for example, T-cells, B-cells, or NK-cells). HSCs have a heterogeneous population in which three classes of stem cells exist, which are distinguished by their ratio of lymphoid to myeloid progeny in the blood (L/M).

As used herein, “CD4+ expressing T-cell,” or “CD4+ T-cell,” are used synonymously throughout, is also known as T helper cells, which play an important role in the immune system, and in the adaptive immune system. CD4+ T-cells also help the activity of other immune cells by releasing T-cell cytokines. These cells help, suppress or regulate immune responses. They are essential in B cell antibody class switching, in the activation and growth of cytotoxic T-cells, and in maximizing bactericidal activity of phagocytes, such as macrophages. CD4+ expressing T-cells make some cytokines, however the amounts of cytokines made by CD4+ T-cells are not at a concentration that promotes, improves, contributes to, or induces engraftment fitness. As described herein, “CD4+ T-cells” are mature T helper-cells that play a role in the adaptive immune system.

As used herein, “CD8+ expressing T-cell” or “CD8+ T-cell,” are used synonymously throughout, is also known as a TC, cytotoxic T lymphocyte, CTL, T-killer cell, cytolytic T-cell or killer T-cell. As described herein, CD8+ T-cells are T-lymphocytes that can kill cancer cells, virally infected cells, or damaged cells. CD8+ T-cells express T-cell receptors (TCRs) that can recognize a specific antigen. CD8+ T-cells express CD8 on the surface. CD8+ expressing T-cells make some cytokines, however the amounts of cytokines made by CD8+ T-cells are not at a concentration that promotes, improves, contributes to, or induces engraftment fitness. “CD8 T-cells” or “killer T-cells” are T-lymphocytes that can kill cancer cells, cells that are infected with viruses or cells that are damaged.

Mature T cells express the surface protein CD4 and are referred to as CD4+ T-cells. CD4+ T-cells are generally treated as having a pre-defined role as helper T-cells within the immune system. For example, when an antigen-presenting cell expresses an antigen on MHC class II, a CD4+ cell will aid those cells through a combination of cell-to-cell interactions (e.g., CD40 and CD40L) and through cytokines. Nevertheless, there are rare exceptions; for example, sub-groups of regulatory T-cells, natural killer cells, and cytotoxic T-cells express CD4. The latter CD4+ expressing T-cell groups are not considered T helper cells.

As used herein, “central memory” T-cell (or “TCM”) refers to an antigen experienced CTL that expresses CD62L or CCR-7 and CD45RO on the surface thereof and does not express or has decreased expression of CD45RA as compared to naïve cells. In some embodiments, central memory cells are positive for expression of CD62L, CCR7, CD28, CD127, CD45RO, and/or CD95, and have decreased expression of CD54RA, as compared to naïve cells.

As used herein, “effector memory” T-cell (or “TEM”) refers to an antigen experienced T-cell that does not express or has decreased expression of CD62L on the surface thereof as compared to central memory cells and does not express or has decreased expression of CD45RA as compared to naïve cell. In some embodiments, effector memory cells are negative for expression of CD62L and/or CCR7, as compared to naïve cells or central memory cells, and have variable expression of CD28 and/or CD45RA.

As used herein, “naïve” T-cells refers to a non-antigen experienced T lymphocyte that expresses CD62L and/or CD45RA, and/or does not express CD45RO− as compared to central or effector memory cells. In some embodiments, naïve CD8+ T lymphocytes are characterized by the expression of phenotypic markers of naïve T-cells including CD62L, CCR7, CD28, CD127, or CD45RA.

As used herein, “effector” “TE” T-cells refers to a antigen experienced cytotoxic T lymphocyte cells that do not express or have decreased expression of CD62L, CCR7, CD28, and are positive for granzyme B or perforin or both, as compared to central memory or naïve T-cells.

As used herein, “protein” has its plain and ordinary meaning when read in light of the specification, and includes, for example, a macromolecule comprising one or more polypeptide chains. A protein can therefore comprise of peptides, which are chains of amino acid monomers linked by peptide (amide) bonds, formed by any one or more of the amino acids. A protein or peptide can contain at least two amino acids, and no limitation is placed on the maximum number of amino acids that can comprise the protein or peptide sequence. Without being limiting, the amino acids are, for example, arginine, histidine, lysine, aspartic acid, glutamic acid, serine, threonine, asparagine, glutamine, cysteine, cystine, glycine, proline, alanine, valine, hydroxyproline, isoleucine, leucine, pyrolysine, methionine, phenylalanine, tyrosine, tryptophan, ornithine, S-adenosylmethionine, or selenocysteine. A protein can also comprise non-peptide components, such as carbohydrate groups, for example. Carbohydrates and other non-peptide substituents can be added to a protein by the cell in which the protein is produced and will vary with the type of cell. Proteins are defined herein in terms of their amino acid backbone structures; substituents such as carbohydrate groups are generally not specified, but can be present nonetheless.

As used herein, “propagating cells” or propagation refers to steps to allow proliferation, expansion, growth and reproduction of cells. For example, cultures of CD8+ T-cells and CD4+ T-cells can typically be incubated under conditions that are suitable for the growth and proliferation of T lymphocytes. In some alternatives of the method of making genetically modified T-cells, which have a chimeric antigen receptor, the CD4+ expressing T-cells are propagated for at least 1 day and may be propagated for 20 days, such as 1, 2, 3, 4, 5, 6, 7, 8, 9, 10, 11, 12, 13, 14, 15, 16, 17, 18, 19, or 20 days or for a period that is within a range defined by any two of the aforementioned time periods. In some alternatives of the method of making genetically modified T-cells, which have a chimeric antigen receptor, the CD8+ expressing T-cells are propagated for at least 1 day and may be propagated for 20 days, such as 1, 2, 3, 4, 5, 6, 7, 8, 9, 10, 11, 12, 13, 14, 15, 16, 17, 18, 19, or 20 days or for a period that is within a range defined by any two of the aforementioned time periods.

In another alternative, the expansion method or propagation can further comprise adding anti-CD3 and/or anti CD28 antibody to the culture medium (e.g., at a concentration of at least 0.5 ng/ml). In another alternative, the method of making genetically modified T-cells, which have a chimeric antigen receptor method can further comprise adding IL-2, IL-15, or IL-21 or any combination thereof to the culture medium (e.g., wherein the concentration of IL-2 is at least 10 units/ml). In another alternative, the method of making genetically modified T-cells, which have a chimeric antigen receptor method can further comprise adding IL-7, IL-15, or IL21 or any combination thereof to the culture medium (e.g., wherein the concentration of IL-2 is at least 10 units/ml). After isolation of T lymphocytes, both cytotoxic and helper T lymphocytes can be sorted into naïve, memory, and effector T-cell subpopulations either before or after expansion.

“Subject” or “patient,” as described herein, refers to any organism upon which the embodiments described herein may be used or administered, e.g., for experimental, diagnostic, prophylactic, and/or therapeutic purposes. Subjects or patients include, for example, animals. In some embodiments, the subject is mice, rats, rabbits, non-human primates, or humans. In some embodiments, the subject is a cow, sheep, pig, horse, dog, cat, primate or a human.

“Cancer,” as described herein, is a group of diseases involving abnormal cell growth with the potential to invade or spread to other parts of the body. Subjects that can be addressed, treated, or their disease being ameliorated using the methods described herein include subjects identified or selected as having cancer, including but not limited to colon, lung, liver, breast, renal, prostate, ovarian, skin (including melanoma), bone, leukemia, multiple myeloma, or brain cancer, etc. Such identification and/or selection can be made by clinical or diagnostic evaluation. In some embodiments, the tumor associated antigens or molecules are known, such as melanoma, breast cancer, brain cancer, squamous cell carcinoma, colon cancer, leukemia, myeloma, or prostate cancer. Examples of cancers that can be treated, inhibited, or ameliorated using one or more of the methods described herein include but are not limited to B cell lymphoma, breast cancer, brain cancer, prostate cancer, and/or leukemia. In some embodiments, one or more oncogenic polypeptides are associated with kidney, uterine, colon, lung, liver, breast, renal, prostate, ovarian, skin (including melanoma), bone, brain cancer, adenocarcinoma, pancreatic cancer, chronic myelogenous leukemia or leukemia.

In some embodiments, a method of treating, ameliorating, or inhibiting one or more of the aforementioned cancers in a subject is provided. In some embodiments, the cancer is breast, ovarian, lung, pancreatic, prostate, melanoma, renal, pancreatic, glioblastoma, neuroblastoma, medulloblastoma, sarcoma, liver, colon, skin (including melanoma), bone or brain cancer. In some embodiments, the subject that receives one of the therapies set forth herein and is also selected to receive an additional cancer therapy, which can include a cancer therapeutic, radiation, chemotherapy, or a cancer therapy drug. In some embodiments, the cancer therapy drug provided comprises Abiraterone, Alemtuzumab, Anastrozole, Aprepitant, Arsenic trioxide, Atezolizumab, Azacitidine, Bevacizumab, Bleomycin, Bortezomib, Cabazitaxel, Capecitabine, Carboplatin, Cetuximab, Chemotherapy drug combinations, Cisplatin, Crizotinib, Cyclophosphamide, Cytarabine, Denosumab, Docetaxel, Doxorubicin, Eribulin, Erlotinib, Etoposide, Everolimus, Exemestane, Filgrastim, Fluorouracil, Fulvestrant, Gemcitabine, Imatinib, Imiquimod, Ipilimumab, Ixabepilone, Lapatinib, Lenalidomide, Letrozole, Leuprolide, Mesna, Methotrexate, Nivolumab, Oxaliplatin, Paclitaxel, Palonosetron, Pembrolizumab, Pemetrexed, Prednisone, Radium-223, Rituximab, Sipuleucel-T, Sorafenib, Sunitinib, Talc Intrapleural, Tamoxifen, Temozolomide, Temsirolimus, Thalidomide, Trastuzumab, Vinorelbine or Zoledronic acid.

Some embodiments include polypeptide sequences or conservative variations thereof, such as conservative substitutions in a polypeptide sequence. In some embodiments, “conservative amino acid substitution” refers to amino acid substitutions that substitute functionally equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide. Substitutions that are charge neutral and which replace a residue with a smaller residue may also be considered “conservative substitutions” even if the residues are in different groups (e.g., replacement of phenylalanine with the smaller isoleucine). Families of amino acid residues having similar side chains have been defined in the art. Several families of conservative amino acid substitutions are shown in TABLE 1.

TABLE 1

| Family | Amino Acids |
|---|---|
| non-polar | Trp, Phe, Met, Leu, Ile, Val, Ala, Pro |
| uncharged polar | Gly, Ser, Thr, Asn, Gln, Tyr, Cys |
| acidic/negatively charged | Asp, Glu |
| basic/positively charged | Arg, Lys, His |
| Beta-branched | Thr, Val, Ile |
| residues that influence | Gly, Pro |
| chain orientation | |
| aromatic | Trp, Tyr, Phe, His |

### Certain Polynucleotides

Some embodiments of the methods and compositions provided herein include a polynucleotide encoding a chimeric polypeptide, such as a chimeric polypeptide comprising a T cell factor 1 (TCF1) domain linked to a pi-catenin domain. In some embodiments, the polynucleotide comprises a first nucleic acid encoding the TCF1 domain; and a second nucleic acid encoding the β-catenin domain.

In some embodiments, the TCF1 domain comprises TCF1 isoform 4S. In some embodiments, the TCF1 domain comprises an amino acid sequence having a percentage sequence identity to the amino acid sequence of SEQ ID NO:02 of at least 90%, 91%, 92%, 93%, 94%, 95%, 96%, 97%, 98%, or 99/o or within a range defined by any two of the aforementioned percentages. In some embodiments, the TCF1 domain comprises or consists of the amino acid sequence of SEQ ID NO:02.

In some embodiments, the β-catenin domain comprises a β-catenin transactivation domain. In some embodiments, the β-catenin transactivation domain comprises an amino acid sequence having a percentage sequence identity to the amino acid sequence of SEQ ID NO:04 of at least 90%, 91%, 92%, 93%, 94%, 95%, 96%, 97%, 98%, or 99% or within a range defined by any two of the aforementioned percentages. In some embodiments, the p-catenin transactivation domain comprises or consists of the amino acid sequence of SEQ ID NO:04.

In some embodiments, the TCF1 domain is linked to the β-catenin transactivation domain via a linker. In some embodiments, the linker has a length in a range from 2 residues to 20 residues, such as 2, 3, 4, 5, 6, 7, 8, 9, 10, 11, 12, 13, 14, 15, 16, 17, 18, 19, or 20 residues or a length that is within a range defined by any two of the aforementioned lengths. In some embodiments, the linker has a length of 10 residues. In some embodiments, the linker comprises the amino acid sequence of SEQ ID NO:03.

In some embodiments, the chimeric polypeptide comprises an amino acid sequence having a percentage sequence identity to the amino acid sequence of SEQ ID NO:01 of at least 90°/%, 91%, 92%, 93%, 94%, 95%, 96%, 97%, 98%, or 99% or within a range defined by any two of the aforementioned percentages. In some embodiments, the chimeric polypeptide comprises or consists of the amino acid sequence of SEQ ID NO:01.

In some embodiments, the polynucleotide also includes a third nucleic acid encoding a cell surface selectable marker. In some embodiments, the cell surface selectable marker is selected from a truncated HER2 (Her2tG) polypeptide, a truncated EGFR (EGFRt) polypeptide, or a truncated CD19 (CD19t). In some embodiments, the second nucleic acid and the third nucleic acid are linked via a ribosomal skip sequence. In some embodiments, the ribosomal skip sequence is selected from P2A, T2A, E2A or F2A.

In some embodiments, the polynucleotide also includes a constitutive promoter operably linked to the first nucleic acid. In some embodiments, the constitutive promoter comprises an EF1α promoter. In some embodiments, the polynucleotide also includes an inducible promoter operably linked to the first nucleic acid.

In some embodiments, the polynucleotide also includes an inducible cytotoxic gene. In some embodiments, the cytotoxic gene encodes a protein selected from a thymidine kinase, thymidine kinase fused to thymidylate kinase, oxidoreductase, deoxycytidine kinase, uracil phosphoribosyltransferase, cytosine deaminase, or cytosine deaminase fused to uracil phosphoribosyltransferase. In some embodiments, the cytotoxic gene encodes a thymidine kinase.

Some embodiments of the methods and compositions provided herein include a vector comprising any one of the polynucleotides provided herein. In some embodiments, the vector comprises a viral vector. In some embodiments, the vector is selected from a lentiviral vector, an adeno-associated viral vector, or an adenoviral vector. In some embodiments, the vector comprises a lentiviral vector.

Some embodiments of the methods and compositions provided herein include a polypeptide encoded by any one of the polynucleotides provided herein. TABLE 2 lists certain amino acid and nucleotide sequences useful with certain embodiments.

TABLE 2

| Feature | Sequence |
|---|---|
| Full-length CA-TCF1 | MYKETVYSAFNLLMHYPPPSGAGQHPQPQPPLHKANQPPHG |
| SEQ ID NO: 01 | VPQLSLYEHFNSPHPTPAPADISQKQVHRPLQTPDLSGFYSLT |
| | SGSMGQLPHTVSWFTHPSLMLGSGVPGHPAAIPHPAIVPPSG |
| | KQELQPFDRNLKTQAESKAEKEAKKPTIKKPLNAFMLYMKE |
| | MRAKVIAECTLKESAAINQILGRRWHALSREEQAKYYELAR |
| | KERQLHMQLYPGWSARDNYGKKKRRSREKHQESTTETNWP |
| | RELKDGNGQESLSMSSSSSPAGGGGSGGGGSDLGLDIGAQGE |
| | PLGYRQDDPSYRSFHSGGYGQDALGMDPMMEHEMGGHHP |
| | GADYPVDGLPDLGHAQDLMDGLPPGDSNQLAWFDTDL |
| TCF7 domain | MYKETVYSAFNLLMHYPPPSGAGQHPQPQPPLHKANQPPHG |
| (isoform 4S) | VPQLSLYEHFNSPHPTPAPADISQKQVHRPLQTPDLSGFYSLT |
| SEQ ID NO: 02 | SGSMGQLPHTVSWFTHPSLMLGSGVPGHPAAIPHPAIVPPSG |
| | KQELQPFDRNLKTQAESKAEKEAKKPTIKKPLNAFMLYMKE |
| | MRAKVIAECTLKESAAINQILGRRWHALSREEQAKYYELAR |
| | KERQLHMQLYPGWSARDNYGKKKRRSREKHQESTTETNWP |
| | RELKDGNGQESLSMSSSSSPA |
| Glycine serine linker | GGGGSGGGGS |
| SEQ ID NO: 03 | |
| β-catenin trans- | DLGLDIGAQGEPLGYRQDDPSYRSFHSGGYGQDALGMDPM |
| activation domain | MEHEMGGHHPGADYPVDGLPDLGHAQDLMDGLPPGDSNQL |
| (amino acids 688-774) | AWFDTDL |
| SEQ ID NO: 04 | |
| Full-length CA-TCF1 | ATGTACAAAGAGACCGTTTATTCCGCGTTTAACCTCTTGAT |
| SEQ ID NO: 05 | GCATTACCCTCCACCCTCTGGTGCCGGACAACACCCACAA |
| | CCGCAACCTCCCTTGCACAAAGCTAATCAACCGCCGCATG |
| | GCGTCCCGCAACTCTCTCTCTATGAACACTTCAACTCTCCA |
| | CACCCAACCCCCGCACCTGCGGACATTTCTCAAAAACAGG |
| | TTCATCGACCTTTGCAAACACCTGACCTGTCAGGCTTTTAC |
| | TCACTGACTAGCGGTTCCATGGGTCAATTGCCACACACAG |
| | TAAGCTGGTTCACGCATCCCTCACTCATGCTTGGGTCCGGC |
| | GTCCCAGGACACCCCGCGGCCATTCCCCATCCTGCCATCG |
| | TTCCGCCATCCGGTAAACAGGAACTGCAGCCATTTGACCG |
| | GAATCTCAAGACGCAAGCAGAGTCCAAGGCTGAGAAGGA |
| | GGCTAAGAAGCCCACGATAAAAAAACCGTTGAATGCGTTT |
| | ATGTTGTACATGAAAGAGATGAGGGCTAAGGTAATAGCG |
| | GAATGCACATTGAAGGAGTCCGCTGCCATCAACCAGATAC |
| | TGGGTAGACGATGGCATGCGTTGTCACGCGAGGAGCAAG |
| | CGAAATATTATGAGCTGGCTAGAAAGGAGAGGCAGCTCC |
| | ACATGCAATTGTATCCCGGTTGGAGCGCGAGGGACAACTA |
| | CGGCAAGAAAAAGAGACGGTCTAGGGAAAAACACCAAGA |
| | AAGTACTACGGAGACGAATTGGCCCAGGGAACTTAAGGA |
| | CGGCAATGGGCAAGAATCTCTTAGTATGTCATCTAGCAGC |
| | TCTCCGGCCGGTGGCGGAGGTTCCGGCGGAGGCGGGTCTG |
| | ATCTGGGCCTTGACATAGGAGCCCAAGGGGAACCGCTTGG |
| | GTATCGACAAGATGATCCTTCATACCGGAGTTTCCATTCTG |
| | GAGGTTATGGGCAAGACGCCCTTGGTATGGACCCTATGAT |
| | GGAGCACGAGATGGGCGGCCATCACCCTGGAGCTGATTAC |
| | CCAGTCGATGGCTTGCCTGACCTGGGCCATGCCCAGGACC |
| | TTATGGATGGCCTCCCACCCGGGGATTCCAACCAGCTGGC |
| | GTGGTTCGACACTGACCTT |
| TCF7 domain | ATGTACAAAGAGACCGTTTATTCCGCGTTTAACCTCTTGAT |
| (Isoform 4S) | GCATTACCCTCCACCCTCTGGTGCCGGACAACACCCACAA |
| SEQ ID NO: 06 | CCGCAACCTCCCTTGCACAAAGCTAATCAACCGCCGCATG |
| | GCGTCCCGCAACTCTCTCTCTATGAACACTTCAACTCTCCA |
| | CACCCAACCCCCGCACCTGCGGACATTTCTCAAAAACAGG |
| | TICATCGACCTTTGCAAACACCTGACCTGTCAGGCTTTTAC |
| | TCACTGACTAGCGGTTCCATGGGTCAATTGCCACACACAG |
| | TAAGCTGGTTCACGCATCCCTCACTCATGCTTGGGTCCGGC |
| | GTCCCAGGACACCCCGCGGCCATTCCCCATCCTGCCATCG |
| | TTCCGCCATCCGGTAAACAGGAACTGCAGCCATTTGACCG |
| | GAATCTCAAGACGCAAGCAGAGTCCAAGGCTGAGAAGGA |
| | GGCTAAGAAGCCCACGATAAAAAAACCGTTGAATGCGTTT |
| | ATGTTGTACATGAAAGAGATGAGGGCTAAGGTAATAGCG |
| | GAATGCACATTGAAGGAGTCCGCTGCCATCAACCAGATAC |
| | TGGGTAGACGATGGCATGCGTTGTCACGCGAGGAGCAAG |
| | CGAAATATTATGAGCTGGCTAGAAAGGAGAGGCAGCTCC |
| | ACATGCAATTGTATCCCGGTTGGAGCGCGAGGGACAACTA |
| | CGGCAAGAAAAAGAGACGGTCTAGGGAAAAACACCAAGA |
| | AAGTACTACGGAGACGAATTGGCCCAGGGAACTTAAGGA |
| | CGGCAATGGGCAAGAATCTCTTAGTATGTCATCTAGCAGC |
| | TCTCCGGCC |
| Glycine serine linker | GGTGGCGGAGGTTCCGGCGGAGGCGGGTCT |
| SEQ ID NO: 07 | |
| β-Catenin trans- | GATCTGGGCCTTGACATAGGAGCCCAAGGGGAACCGCTTG |
| activation domain | GGTATCGACAAGATGATCCTTCATACCGGAGTTTCCATTCT |
| SEQ ID NO: 08 | GGAGGTTATGGGCAAGACGCCCTTGGTATGGACCCTATGA |
| | TGGAGCACGAGATGGGCGGCCATCACCCTGGAGCTGATTA |
| | CCCAGTCGATGGCTTGCCTGACCTGGGCCATGCCCAGGAC |
| | CTTATGGATGGCCTCCCACCCGGGGATTCCAACCAGCTGG |
| | CGTGGTTCGACACTGACCTT |
| T2A | GGCGGCGGAGAGGGCAGAGGAAGTCTTCTAACATGCGGT |
| SEQ ID NO: 09 | GACGTGGAGGAGAATCCCGGCCCT |

### Certain Cells

Some embodiments of the methods and compositions provided herein include a cell or cell population comprising any one of the polynucleotides provided herein, or any one of the chimeric proteins comprising a TCF1 domain and a β-catenin domain provided herein.

In some embodiments, the cell also includes a fourth nucleic acid encoding a chimeric antigen receptor (CAR), and/or a CAR protein. In some embodiments, the CAR is capable of specifically binding to a target antigen expressed by a cancer cell. In some embodiments, the CAR is a bi-specific CAR.

In some embodiments, the cell is a T cell. In some embodiments, the cell is derived from a CD4+ T cell, a CD8+ T cell, a precursor T cell, or a hematopoietic stem cell. In some embodiments, the CD8+ T cell is a CD8+ cytotoxic T lymphocyte cell selected from the group consisting of a naïve CD8+ T cell, a central memory CD8+ T cell, an effector memory CD8+ T cell, and a bulk CD8+ T cell. In some embodiments, the CD4+ cell is a CD4+ helper T lymphocyte cell selected from the group consisting of a naïve CD4+ T cell, a central memory CD4+ T cell, an effector memory CD4+ T cell, and a bulk CD4+ T cell.

In some embodiments, the cell is a primary cell. In some embodiments, the cell is mammalian. In some embodiments, the cell is human. In some embodiments, the cell is ex vivo. In some embodiments, the cell is allogenic to a subject, preferably a human, and in other embodiments, the cell is autologous to a subject, preferably a human.

Some embodiments of the methods and compositions provided herein include pharmaceutical compositions comprising any one of the cells provided herein and a pharmaceutically acceptable excipient.

### Certain Therapeutic Methods

Some embodiments of the methods and compositions provided herein relate to therapies. Some embodiments of the methods and compositions provided herein relate to methods of treating, ameliorating or inhibiting a disease, such as a cancer, or a disorder stemming therefrom in a subject, comprising administering any one of the cells provided herein to the subject in need thereof. In some embodiments, the cell comprises a CAR. In some embodiments, the disease is a cancer that results from cancer cells having a target antigen, wherein the CAR is capable of specifically binding to the target antigen. For example, a target antigen can include an antigen expressed at higher levels by a cancer cell than by a non-cancer cell, or an antigen absent from a non-cancer cell. In some embodiments, the cancer is selected from a solid tumor such as a colon cancer, breast cancer, ovarian cancer, lung cancer, pancreatic cancer, prostate cancer, melanoma, renal cancer, pancreatic cancer, brain cancer, glioblastoma, neuroblastoma, medulloblastoma, sarcoma, bone cancer, or liver cancer, or a non-solid tumor, such as a leukemia, or a multiple myeloma. In some embodiments, the cell is allogenic to the subject. In some embodiments, the cell is autologous to a subject. In some embodiments, the cell is not autologous to the subject that receives the cell. In some embodiments, the subject is mammalian. In some embodiments, the subject is human. Some embodiments of the methods and compositions provided herein relate to any one of the cells or cell populations provided herein for use in treating, ameliorating or inhibiting a disorder such as a cancer in a subject, preferably a subject selected or identified to receive any one or more of the cells or cell populations described herein, e.g., such subjects can be selected or identified on the basis of clinical and/or diagnostic evaluation for the particular disease, such as a cancer, for which the cells or cell populations are being administered.

### Certain Populations of Cells

Some embodiments of the methods and compositions provided herein relate to populations of cells comprising a chimeric protein comprising a TCF1 domain and a β-catenin domain. In some embodiments, some such populations have an increased level of a memory cell marker, or activity of a memory cell as compared to the level of the memory cell marker, or the activity of a memory cell in a population of cells lacking the chimeric protein or the polynucleotide encoding such a chimeric protein. In some embodiments, the memory T cell marker is selected from CCR7, CD62L, CD127, CD45RO, CD44, CD27, CD28, CD95, CXCR3, or LFA-1.

Some embodiments of the methods and compositions provided herein include methods of preparing a population of cells comprising introducing any one of the polynucleotides provided herein into a cell to obtain a transduced cell; and culturing the transduced cell to obtain a population of cells expressing a chimeric protein comprising a TCF1 domain and a pi-catenin domain, which may or may not be presented as part of a CAR. In some embodiments, the population of cells having a chimeric protein comprising a TCF1 domain and a β-catenin domain exhibits an increased level of a memory T cell marker and/or memory T cell activity as compared to a population of cells lacking the polynucleotide.

In some embodiments, the T cell is derived from a CD4+ T cell, a CD8+ T cell, a precursor T cell, or a hematopoietic stem cell. In some embodiments, the CD8+ T cell is a CD8+ cytotoxic T lymphocyte cell selected from the group consisting of a naïve CD8+ T cell, a central memory CD8+ T cell, an effector memory CD8+ T cell, and a bulk CD8+ T cell. In some embodiments, the CD4+ cell is a CD4+ helper T lymphocyte cell selected from the group consisting of a naïve CD4+ T cell, a central memory CD4+ T cell, an effector memory CD4+ T cell, and a bulk CD4+ T cell. In some embodiments, the cell is a primary cell. In some embodiments, the cell is mammalian. In some embodiments, the cell is human. In some embodiments, the cell is ex vivo.

### EXAMPLES

### Example 1—Generation of TCF1-β Catenin Transgene

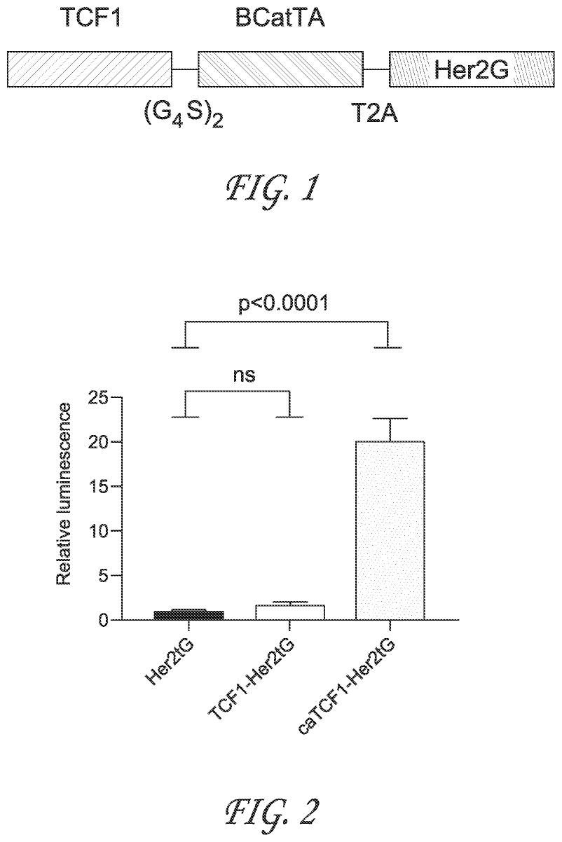

A constitutively active (CA) T cell factor 1 (TCF1)-β-catenin (CA-TCF1) transgene was assembled by fusing a 4S isoform of a human T cell factor 7 (TCF7) gene with a transactivation domain of a human β-catenin gene with an intervening (Glycine4Serine)2 linker (SEQ ID NO:03). FIG. 1 depicts a schematic view of a transgene encoding a TCF1 domain, a glycine-serine linker (G₄S)₂, a β catenin transactivator domain (BCatTA), a T2A ribosome skip sequence, and truncated HER2 polypeptide (Her2tG). The truncated HER2 polypeptide is a cell surface marker. Amino acid sequences of TCF7 and β-catenin were accessed using an online protein sequence database UniProt. A DNA sequence of the fully assembled amino acid sequence was generated using the Integrated DNA Technology (IDT) optimization tool, which was set to optimize codons for human expression. Finally, the DNA sequence was synthesized by IDT and inserted via Gibson assembly methods into an epHIV7.2 lentiviral transfer plasmid for gene delivery. TABLE 1 lists certain nucleotide and amino acid sequences that were used in the construction of the transgene, and vectors including the transgene.

### Example 2—In Vitro Activity of TCF1-β-Catenin (CA-TCF1) Transgene

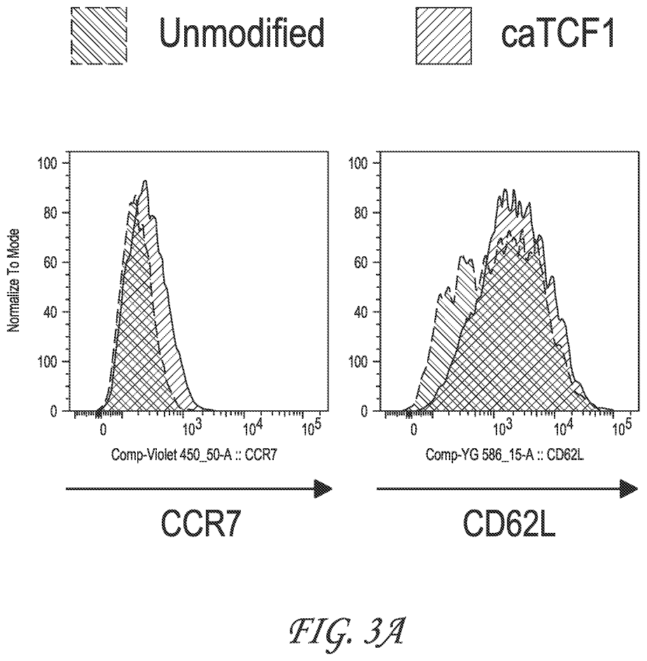

To test the transcriptional activity of the CA-TCF1 protein, human embryonic kidney (HEK) 293T cells were transiently transfected with a reporter plasmid encoding a luciferase protein subject to transcriptional regulation by a series of consensus TCF transcriptional responsive elements (Addgene Cat. #12456). Simultaneously, HEK cells were transfected with a lentiviral transfer plasmid encoding either a surface marker gene (Her2tG), an unmodified full-length TCF1 gene, or the CA-TCF1 gene (FIG. 2). Twenty-four hours post-transfection, the HEK cells were removed from culture vessels using trypsin and seeded into opaque flat-bottom 96-well plates for later analysis. Forty-eight hours post-transfection, D-luciferin was added to the culture vessels and luminescence was measured using a Perkin Elmer Victor 3 plate reader.

CA-TCF1, unlike native TCF1, had intrinsic transcriptional activity. The transcriptional activity of CA-TCF1 was examined using a reporter plasmid encoding luciferase under the regulation of TCF1-responsive elements. When this reporter construct was introduced into HEK cells alongside a plasmid encoding the native form of TCF1 (TCF1-Her2tG), there was no significant increase in luciferase expression compared to a surface marker control plasmid (Her2tG) (FIG. 2). However, the introduction of the CA-TCF1 transgene resulted in a ˜20-fold increase in luciferase expression, which demonstrated that CA-TCF1 was able to activate transcription of TCF-dependent genes, while native TCF1 failed to activate transcription without supplemental expression of β-catenin.

### Example 3—In Vitro Activity of TCF1-β Catenin Transgene in CD8+ T Cells

Recombinant lentivirus was generated by transiently transfecting HEK 293T producer cells with lentiviral packaging plasmids alongside a transfer plasmid encoding the CA-TCF1 transgene. Transfection was performed using Lipofectamine 2000 (Life Technologies, Cat. #11668-500). Four days after transfection, lentivirus was isolated from the 293T cell culture supernatant via ultracentrifugation and stored at −80° C. until the day of transduction.

CD8+ T cells were isolated from human peripheral blood mononuclear cells (PBMCs) by magnetic activated cell sorting with a CD8+ T cell isolation kit (Miltenyi Biotech, Cat. #130-096-495). The cells were immediately subjected to a bead-based CD3/CD28 stimulation using Dynabeads (Thermo Fisher Scientific, Cat. #11131D) at a bead to cell ratio of 1:1. T cell culture media consisted of RPMI 1640 (Gibco, Cat. #22400-089) supplemented with 10% FBS (Hyclone, Cat. #SH30071.03), 2 mM L-glutamine (Gibco, 25030-081), 50 U/mL IL-2 (Chiron, Cat. #53905-991-01) and 0.5 ng/mL IL-15 (Miltenyi, Cat. #130-095-765) throughout the culture period. Two days post-stimulation, cells were transduced with lentivirus housing the CA-TCF1 transgene.

Twenty-one days post stimulation, T cells were subjected to a second stimulation using Dynabeads in the absence of cytokine. Then cells were analyzed via flow cytometry for surface expression of CD62L and CCR7, two markers associated with the central memory T cell phenotype (FIG. 3A, FIG. 3B).

CA-TCF1 promoted the expression of memory-associated surface markers in CD8 T cells. Endogenous TCF1 promotes the memory T cell characteristics, and we examined expression of surface markers associated with memory T cells. When stimulated using CD3/CD28 beads, CA-TCF1-expressing T cells showed higher expression of CD62L and CCR7, two surface markers of central memory T cells, when compared to unmodified T cells (FIG. 3).

### Example 4—In Vivo Activity of CA-TCF1 in a Subcutaneous Tumor Model

To determine the effect of CA-TCF1 on CAR T cell potency in vivo, a xenograft tumor mouse model is employed. The study is conducted using immunocompromised NSG mice, which allow for engraftment of human tumor cells and subsequent treatment with human CAR T cells. Human T cells are modified to express an anti-B7H3 CAR and firefly luciferase. The luciferase enables bioluminescent imaging of T cells in vivo, and to track T cell presence and expansion. Tumor volume of subcutaneous tumors can also be readily measured manually. The T cell experimental group of interest is also modified to express CA-TCF1.

Mice are subcutaneously injected with a human tumor cell line, such as the neuroblastoma cell line Be2, which expresses B7H3. Five days after injection of tumor cells, mice are injected with the modified CAR T cells via the tail vein. The mice are monitored for: (1) tumor volume progression by manual caliper measurements; (2) T cell expansion and persistence via bioluminescent imaging; and (3) mouse survival.

Mice treated with T cells also containing the CA-TCF1 transgene will have a reduced tumor progression, a greater percent tumor clearance, a greater CAR T cell presence over time, and/or greater survival, as compared to mice treated with T cells lacking the CA-TCF1 transgene.

### Example 5—In Vivo Activity of CA-TCF1 in a Systemic Tumor Model

The activity of CA-TCF1 is tested using a systemic tumor model. Human CAR T cells with and without the CA-TCF1 transgene are prepared. NSG mice are injected intravenously with tumor cells such as the human Raji lymphoma cell line, modified to express the firefly luciferase transgene. The CAR of the human CAR T cells specifically binds to an antigen expressed by the Raji cells. The human CAR T cells are administered to the mice by intravenous injection. The mice are monitored for tumor progression by bioluminescent imaging and mouse survival. Longitudinal T cell presence is quantified by retro-orbital bleeds followed by flow cytometry to quantify the presence of circulating human T cells.

In this systemic model, mice treated with CA-TCF1-equipped CAR T cells will show slower tumor progression, a greater percent tumor clearance, a greater T cell presence over time, and/or a greater survival, as compared to mice treated with T cells lacking CA-TCF1 supplementation. The CA-TCF1 promotes T cell characteristics that may encourage greater engraftment in the lymphatic microenvironment than in the subcutaneous tumor microenvironment.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A polynucleotide encoding a chimeric polypeptide comprising:
a first nucleic acid encoding a T cell factor 1 (TCF1) domain; and
a second nucleic acid encoding a β-catenin transactivation domain.

2-55. (canceled)
